(19) United States
(12) Patent Application Publication
(10) Pub. No.: US 2025/0349094 A1
(43) Pub. Date: Nov. 13, 2025

(54) AUGMENTED REALITY SURGICAL SYSTEMS AND METHODS USING DEPTH SENSING

(71) Applicant: AUGMEDICS LTD., Yokneam Illit (IL)

(72) Inventors: Nissan Elimelech, Beerotaim (IL); Stuart Wolf, Yokneam (IL); Nitzan Krasney, Haifa (IL); Monica Marie Kuhnert, Chicago, IL (US)

(21) Appl. No.: 19/276,148

(22) Filed: Jul. 22, 2025

Related U.S. Application Data
(63) Continuation of application No. 18/365,844, filed on Aug. 4, 2023, now Pat. No. 12,417,595.
Application No. 18/365,844 is a continuation of application No. PCT/IB2022/057733, filed on Aug. 18, 2022.
(60) Provisional application No. 63/234,272, filed on Aug. 18, 2021.
(60) Provisional application No. 63/236,244, filed on Aug. 24, 2021.
(60) Provisional application No. 63/236,241, filed on Aug. 24, 2021.
(60) Provisional application No. 63/281,677, filed on Nov. 21, 2021.

(51) Int. Cl. G06T 19/00; A61B 34/10; A61B 34/20; A61B 90/00; G02B 27/01; G06T 7/00; G06T 7/30; G06T 7/521; G06T 7/55; G06T 7/593; G06T 7/62; G06T 7/73; H04N 13/128; H04N 13/156; H04N 13/167; H04N 13/239; H04N 13/344; H04N 13/383; H04N 23/11

(52) U.S. Cl. CPC G06T 19/006; A61B 34/20; A61B 90/37; G02B 27/0172; G06T 7/0016; G06T 7/30; G06T 7/521; G06T 7/55; G06T 7/593; G06T 7/62; G06T 7/73; H04N 13/128; H04N 13/156; H04N 13/167; H04N 13/239; H04N 13/344; H04N 13/383; H04N 23/11; A61B 2034/107; A61B 2090/365; A61B 2090/367; A61B 2090/371; A61B 2090/373; G02B 2027/0134; G02B 2027/0138; G02B 2027/014; G02B 2027/0141; G02B 2027/0178; G06T 2200/04; G06T 2207/10012; G06T 2207/10028; G06T 2207/10081; G06T 2207/20084; G06T 2207/30008; G06T 2207/30012; G06T 2207/30204; G06T 2210/12; G06T 2210/41

(57) ABSTRACT

A system for image-guided surgery, the system comprising: a head-mounted unit comprising: a see-through augmented reality display; a depth sensor configured to generate depth data with respect to a region of interest (ROI) on a body of a patient viewed through the display by a user wearing the head-mounted unit and with respect to a surgical item when the item is placed within a field of view of the depth sensor, wherein the item comprises a first marker containing a predefined pattern disposed on the item; and a processor configured to: process the depth data to identify a shape of the item; compute, a first spatial transformation between a position of the first marker and a location and orientation of the item; track the position of the first marker as the user manipulates item; and generate an image of the item on the display in registration with the ROI.

### CROSS-REFERENCE TO RELATED APPLICATIONS

This application is a continuation of U.S. patent application Ser. No. 18/365,844, filed on Aug. 4, 2023, which is a continuation of International PCT Application PCT/IB2022/057733, filed Aug. 18, 2022, which claims the benefit of: U.S. Provisional Patent Application 63/236,241, filed Aug. 24, 2021; U.S. Provisional Patent Application 63/281,677, filed Nov. 21, 2021; U.S. Provisional Patent Application No. 63/234,272, filed Aug. 18, 2021; and U.S. Provisional Patent Application No. 63/236,244, filed Aug. 24, 2021. The entire content of each of these related applications is incorporated herein by reference.

### FIELD

The present disclosure relates generally to image-guided surgery or intervention, and specifically to systems and methods for use of augmented reality in image-guided surgery or intervention and/or to systems and methods for use in surgical computer-assisted navigation.

### BACKGROUND

Near-eye display devices and systems can be used in augmented reality systems, for example, for performing image-guided surgery. In this way, a computer-generated image may be presented to a healthcare professional who is performing the procedure such that the image is aligned with an anatomical portion of a patient who is undergoing the procedure. Applicant's own work has demonstrated an image of a tool that is used to perform the procedure can also be incorporated into the image that is presented on the head-mounted display. For example, Applicant's prior systems for image-guided surgery have been effective in tracking the positions of the patient's body and the tool (see, for example, U.S. Pat. Nos. 9,928,629, 10,835,296, 10,939,977, PCT International Publication WO 2019/211741, and U.S. Patent Application publication 2020/0163723.) The disclosures of all these patents and publications are incorporated herein by reference.

### SUMMARY

Embodiments of the present disclosure provide improved systems, methods, and software for image-guided surgery. Some embodiments of the systems improve the accuracy of the augmented reality images that are presented on the display and broaden the capabilities of the augmented reality system by employing depth sensing.

In some embodiments, a system for image-guided surgery comprises a head-mounted unit, comprising a see-through augmented reality display and a depth sensor, which is configured to generate depth data with respect to a region of interest (ROI) of a body of a patient that is viewed through the display by a user wearing the head-mounted unit; and a processor, which is configured to receive a three-dimensional (3D) tomographic image of the body of the patient, to compute a depth map of the ROI based on the depth data generated by the depth sensor, to compute a transformation over the ROI so as to register the tomographic image with the depth map, and to apply the transformation in presenting a part of the tomographic image on the display in registration with the ROI viewed through the display.

In some embodiments, the ROI comprises a bone of the body to which an anchoring device is fastened, and wherein the processor is further configured to identify a location of the anchoring device in the depth map, to update the depth map in the course of a surgery, to detect a change in the location of the anchoring device in the updated depth map, and to take a corrective action responsively to the change.

In some embodiments, the corrective action comprises modifying a presentation on the display responsively to the change in the location of the anchoring device.

In some embodiments, the depth map includes a spine of the patient, which is exposed in a surgical procedure, and wherein the processor is configured to compute the transformation by registering the spine in the depth map with the spine appearing in the tomographic image.

In some embodiments, the processor is configured to process the depth data so as to detect a position of a marker that is fixed to the body of the patient, to recognize a location of the head-mounted unit relative to the body based on the detected position, and to position the image presented on the display responsively to the recognized location.

In some embodiments, the processor is configured to process the depth data so as to identify a change in an anatomical structure in the body of the patient during a surgical procedure, and to modify the image presented on the display responsively to the identified change.

In some embodiments, the processor is configured to process the depth data so as to identify an implant inserted into the body of the patient during a surgical procedure, and to modify the image presented on the display responsively to the identified implant.

In some embodiments, the tomographic image comprises a CT scan of the patient, which was performed with an array of radiopaque fiducial markers fixed to the body of the patient, and wherein the processor is configured to identify respective 3D coordinates of the fiducial markers in the depth map and to register the CT scan with the ROI viewed through the display by matching the fiducial markers in the CT to the respective 3D coordinates.

In some embodiments, a system for image-guided surgery comprises a head-mounted unit, comprising: a see-through augmented reality display; and a depth sensor, which is configured to generate depth data with respect to a region of interest (ROI) on a body of a patient that is viewed through the display by a user wearing the head-mounted unit and with respect to a surgical tool when the tool is placed within a field of view of the depth sensor, wherein the tool comprises a shaft and a marker containing a predefined pattern disposed on the tool in a fixed spatial relation to the shaft; and a processor, which is configured to: process the depth data so as to identify a shape of the tool and to compute, responsively to the shape, a spatial transformation between a position of the marker and a location and orientation of the shaft; track the position of the marker as the user manipulates the shaft of the tool within the body, and using the tracked position and the spatial transformation, generate an image of the tool, including the shaft, on the display in registration with the ROI viewed through the display.

In some embodiments, further comprising a tracking sensor, which is disposed on the head-mounted unit in a known spatial relation to the depth sensor and is configured to detect the position of the marker.

In some embodiments, the shaft has a curved shape, and wherein the processor is configured to process the depth data so as to reconstruct a three-dimensional (3D) model of the curved shape, and to generate the image of the tool based on the 3D model.

In some embodiments, the processor is configured to process the depth data so as detect a change in a shape of the tool and to update the image of the tool on the display responsively to the change in the shape.

In some embodiments, the depth sensor is further configured to generate the depth data with respect to a further marker that is attached to the body of the patient, and wherein the processor is configured to apply the depth data in calculating a position of the tool relative to the body.

In some embodiments, the depth sensor is configured to generate further depth data with respect to a hand of a user of the head-mounted unit, and wherein the processor is configured to process the further depth data so as to detect a gesture made by the hand, and to control a function of the system responsively to the detected gesture.

In some embodiments, a system for image-guided surgery, comprises a head-mounted unit, comprising a see-through augmented reality display and a depth sensor, which is configured to generate depth data with respect to a region of interest (ROI) on a body of a patient that is viewed through the display by a user wearing the head-mounted unit and with respect to a surgical implant when the implant is placed within a field of view of the depth sensor, wherein the implant is configured to be mounted on a shaft of a surgical tool and inserted, using the tool, into the body; and a processor, which is configured to process the depth data so as to identify a shape of the implant and to compute, responsively to the shape, a spatial transformation between a position of a marker disposed on the tool and a location and orientation of the implant, to track the position of the marker as the user manipulates the shaft of the tool within the body, and using the tracked position, the spatial transformation, and the identified shape, to generate on the display an image of the implant within the body in registration with the ROI viewed through the display, wherein the marker contains a predefined pattern and is disposed in a fixed spatial relation to the shaft.

In some embodiments, the system further comprising a tracking sensor, which is disposed on the head-mounted unit in a known spatial relation to the depth sensor and is configured to detect the position of the marker.

In some embodiments, the shaft has a curved shape, and wherein the processor is configured to process the depth data so as to reconstruct a three-dimensional (3D) model of the curved shape, and to generate the image of the implant based on the 3D model.

In some embodiments, the processor is configured to process the depth data so as detect a change in a shape of the tool and to update the spatial transformation responsively to the change in the shape.

In some embodiments, a system for image-guided surgery, comprises a head-mounted unit, comprising a see-through augmented reality display and a depth sensor, which is configured to generate depth data with respect to a region of interest (ROI) on a body of a patient, including a bone inside the body, that is viewed through the display by a user wearing the head-mounted unit; and a processor, which is configured to process the depth data generated by the depth sensor so as to identify a first three-dimensional (3D) shape of the bone prior to a surgical procedure on the bone and a second 3D shape of the bone following the surgical procedure, and to generate, based on the first and second 3D shapes, an image showing a part of the bone that was removed in the surgical procedure.

In some embodiments, a method for image-guided surgery comprises using a head-mounted unit that includes a see-through augmented reality display and a depth sensor, generating depth data with respect to a region of interest (ROI) of a body of a patient that is viewed through the display by a user wearing the head-mounted unit; receiving a three-dimensional (3D) tomographic image of the body of the patient; computing a depth map of the ROI based on the depth data generated by the depth sensor; computing a transformation over the ROI so as to register the tomographic image with the depth map; and applying the transformation in presenting a part of the tomographic image on the display in registration with the ROI viewed through the display.

In some embodiments, the ROI comprises a bone of the body to which an anchoring device is fastened, and wherein the method comprises: identifying an initial location of the anchoring device in the depth map; updating the depth map in the course of a surgery; detecting a change in the location of the anchoring device in the updated depth map; and taking a corrective action responsively to the change.

In some embodiments, taking the corrective action comprises modifying a presentation on the display responsively to the change in the location of the anchoring device.

In some embodiments, the depth map includes a spine of the patient, which is exposed in a surgical procedure, and wherein computing the transformation comprises registering the spine in the depth map with the spine appearing in the tomographic image.

In some embodiments, the method further comprises processing the depth data so as to detect a position of a marker that is fixed to the body of the patient; recognizing a location of the head-mounted unit relative to the body based on the detected position; and positioning the image presented on the display responsively to the recognized location.

In some embodiments, the method further comprises processing the depth data so as to identify a change in an anatomical structure in the body of the patient during a surgical procedure; and modifying the image presented on the display responsively to the identified change.

In some embodiments, the method further comprises processing the depth data so as to identify an implant inserted into the body of the patient during a surgical procedure; and modifying the image presented on the display responsively to the identified implant.

In some embodiments, the tomographic image comprises a CT scan of the patient, which was performed with an array of radiopaque fiducial markers fixed to the body of the patient, and wherein computing the transformation comprises identifying respective 3D coordinates of the fiducial markers in the depth map, and registering the CT scan with the ROI viewed through the display by matching the fiducial markers in the CT to the respective 3D coordinates.

In some embodiments, a method for image-guided surgery comprises using a head-mounted unit that includes a see-through augmented reality display and a depth sensor, generating depth data with respect to a region of interest (ROI) on a body of a patient that is viewed through the display by a user wearing the head-mounted unit and with respect to a surgical tool when the tool is placed within a field of view of the depth sensor, wherein the tool comprises a shaft and a marker containing a predefined pattern disposed on the tool in a fixed spatial relation to the shaft; processing the depth data so as to identify a shape of the tool and to compute, responsively to the shape, a spatial transformation between a position of the marker and a location and orientation of the shaft; tracking the position of the marker as the user manipulates the shaft of the tool within the body; and using the tracked position and the spatial transformation, generating an image of the tool, including the shaft, on the display in registration with the ROI viewed through the display.

In some embodiments, the tracking the position comprises detecting the position of the marker using a tracking sensor disposed on the head-mounted unit in a known spatial relation to the depth sensor.

In some embodiments, the shaft has a curved shape, and wherein processing the depth data comprises reconstructing a three-dimensional (3D) model of the curved shape, wherein the image of the tool is generated based on the 3D model.

In some embodiments, processing the depth data comprises detecting a change in a shape of the tool, and wherein generating the image comprises updating the image of the tool on the display responsively to the change in the shape.

In some embodiments, generating the depth data comprises capturing further depth data with respect to a further marker that is attached to the body of the patient, and wherein processing the depth data comprises applying the further depth data in calculating a position of the tool relative to the body.

In some embodiments, generating the depth data comprises capturing further depth data with respect to a hand of a user of the head-mounted unit, and wherein the method comprises processing the further depth data so as to detect a gesture made by the hand, and controlling a function of the head-mounted unit responsively to the detected gesture.

In some embodiments, a method for image-guided surgery comprises using a head-mounted unit that includes a see-through augmented reality display and a depth sensor, generating depth data with respect to a region of interest (ROI) on a body of a patient that is viewed through the display by a user wearing the head-mounted unit and with respect to a surgical implant when the implant is placed within a field of view of the depth sensor, wherein the implant is mounted on a shaft of a surgical tool and inserted, using the tool, into the body; processing the depth data so as to identify a shape of the implant; computing, responsively to the shape, a spatial transformation between a position of a marker disposed on the tool and a location and orientation of the implant, wherein the marker contains a predefined pattern and is disposed in a fixed spatial relation to the shaft; tracking the position of the marker as the user manipulates the shaft of the tool within the body; and using the tracked position, the spatial transformation, and the identified shape, generating on the display an image of the implant within the body in registration with the ROI viewed through the display.

In some embodiments, the method further comprises detecting the position of the marker using a tracking sensor, which is disposed on the head-mounted unit in a known spatial relation to the depth sensor.

In some embodiments, the shaft has a curved shape, and wherein processing the depth data comprises reconstructing a three-dimensional (3D) model of the curved shape, wherein the image of the implant is generated based on the 3D model.

In some embodiments, processing the depth data comprises detecting a change in a shape of the tool, and updating the spatial transformation responsively to the change in the shape.

In some embodiments, a method for image-guided surgery, comprises using a head-mounted unit that includes a see-through augmented reality display and a depth sensor, generating depth data with respect to a region of interest (ROI) on a body of a patient, including a bone inside the body, that is viewed through the display by a user wearing the head-mounted unit; processing the depth data generated by the depth sensor so as to identify a first three-dimensional (3D) shape of the bone prior to a surgical procedure on the bone and a second 3D shape of the bone following the surgical procedure; and generating, based on the first and second 3D shapes, an image showing a part of the bone that was removed in the surgical procedure.

In some embodiments, the surgical procedure involves a bone cut.

In some embodiments, a head-mounted system for image-guided surgery comprises a see-through augmented reality display disposed so as to be viewable by a user over a region of interest (ROI) of a body of a patient; a depth sensor configured to generate depth data with respect to the ROI; and a processor and a memory for storing instructions that, when executed by the processor cause the system to: receive a three-dimensional (3D) tomographic image of the body of the patient; determine a depth map of the ROI based at least in part on the depth data; determine a transformation over the ROI so as to register the 3D tomographic image with the depth map; and display at least a part of the 3D tomographic image on the see-through augmented reality display in registration with the ROI based at least in part on the transformation.

In some embodiments, a method for image-guided surgery comprises a see-through augmented reality display disposed so as to be viewable by a user over a region of interest (ROI) of a body of a patient; a depth sensor configured to generate depth data with respect to the ROI; and a processor and a memory for storing instructions that, when executed by the processor cause the system to: receive a three-dimensional (3D) tomographic image of the body of the patient; determine a depth map of the ROI based at least in part on the depth data; determine a transformation over the ROI so as to register the 3D tomographic image with the depth map; and display at least a part of the 3D tomographic image on the see-through augmented reality display in registration with the ROI based at least in part on the transformation.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

The present invention will be more fully understood from the following detailed description of the embodiments thereof, taken together with the drawings in which:

### DESCRIPTION OF THE DRAWINGS

Non-limiting features of some embodiments of the invention are set forth with particularity in the claims that follow. The following drawings are for illustrative purposes only and show non-limiting embodiments. Features from different figures may be combined in several embodiments.

FIG. 1 is a schematic pictorial illustration showing a system for image-guided surgery, in accordance with an embodiment of the disclosure;

FIG. 2A is a schematic pictorial illustration showing details of a near-eye unit that is used for image-guided surgery, in accordance with an embodiment of the disclosure;

FIG. 2B is a schematic pictorial illustration showing details of a head-mounted unit that is used for image-guided surgery, in accordance with another embodiment of the disclosure;

FIG. 3A is a schematic pictorial illustration showing details of a surgical tool, in accordance with an embodiment of the disclosure;

FIG. 3B is a schematic pictorial illustration showing details of a surgical tool, in accordance with another embodiment of the disclosure;

FIGS. 4A and 4B are flow charts that schematically illustrate methods for image-guided surgery, in accordance with embodiments of the disclosure;

FIGS. 5, 6 and 7 are flow charts that schematically illustrate methods for image-guided surgery, in accordance with further embodiments of the disclosure; and

FIG. 8 is a schematic pictorial illustration showing a system for image-guided surgery, in accordance with an alternative embodiment of the disclosure.

### DETAILED DESCRIPTION

### Overview

Embodiments of the present disclosure that are described herein provide systems, methods and software for image-guided surgery or other intervention, computer assisted navigation and/or stereotactic surgery or other intervention that, inter alia, use depth sensing to enhance the capabilities of an augmented reality display and system. In some embodiments, a head-mounted unit comprises both a see-through augmented reality display and a depth sensor. In some embodiments, the depth sensor generates depth data with respect to a region of interest (ROI) on a body of a patient that is viewed through the display by a user wearing the head-mounted unit. In some embodiments, the system applies the depth data in generating one or more depth maps of the body. Additionally or alternatively, the depth sensor may be applied in generating depth data with respect to implements such as clamps, tools and implants that can be inserted into the body. In some embodiments, using the depth data, the system is able to improve the accuracy of the augmented reality images that are presented on the display and broaden the capabilities of the augmented reality system.

In some embodiments, the term “depth sensor” refers to one or more optical components that are configured to capture a depth map of a scene. For example, in some embodiments, the depth sensor can be a pattern projector and a camera for purposes of structured-light depth mapping. For example, in some embodiments, the depth sensor can be a pair of cameras configured for stereoscopic depth mapping. For example, in some embodiments, the depth sensor can be a beam projector and a detector (or an array of detectors) configured for time-of-flight measurement. Of course the term “depth sensor” as used herein is not limited to the listed examples and can other structure.

### System Description

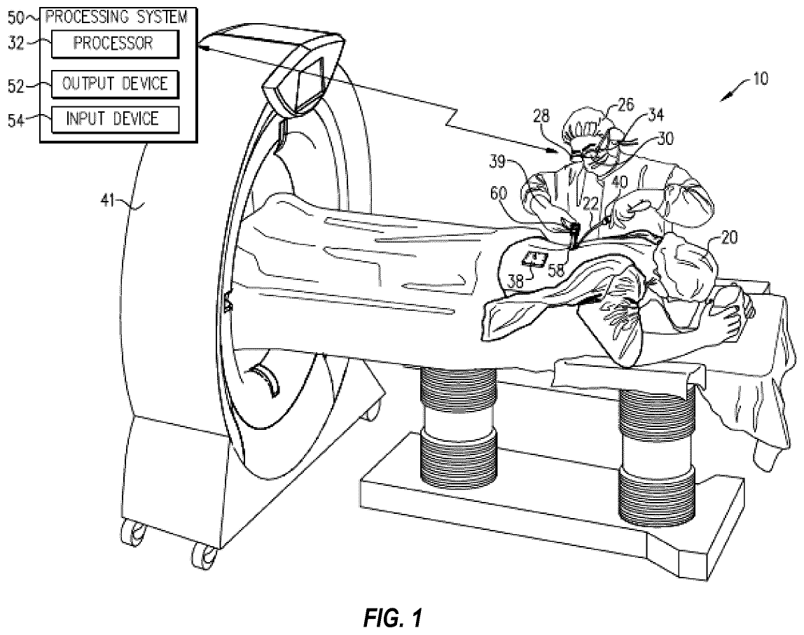

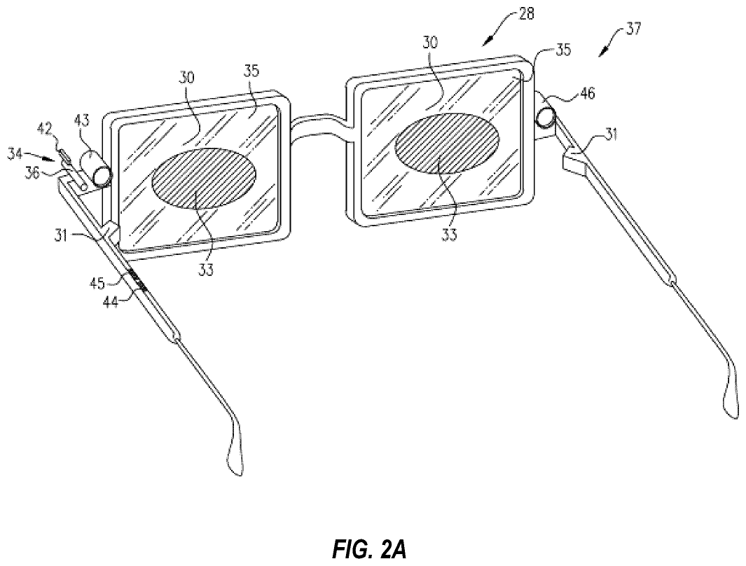

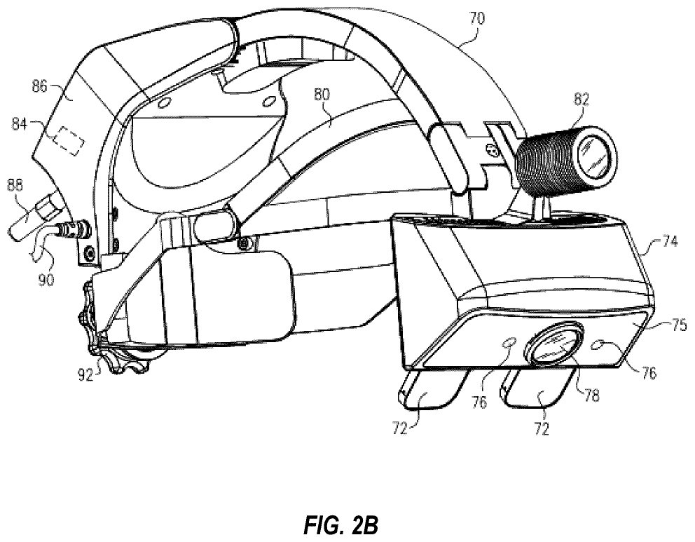

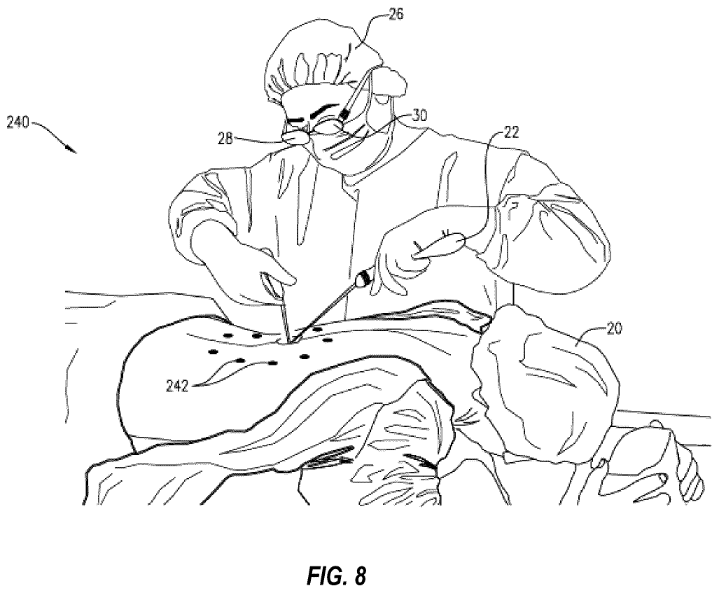

Reference is now made to FIGS. 1 and 2A, which schematically illustrate an exemplary system 10 for image-guided surgery, in accordance with some embodiments of the disclosure. For example, FIG. 1 is a pictorial illustration of the system 10 as a whole, while FIG. 2A is a pictorial illustration of a near-eye unit that is used in the system 10. The near eye unit illustrated in FIGS. 1 and 2A is configured as a head-mounted unit 28. In some embodiments, the near-eye unit can be configured as the head-mounted unit 28 shown in FIG. 8 and as a head-mounted AR display (HMD) unit 70, 100 and described hereinbelow. In FIG. 1, the system 10 is applied in a medical procedure on a patient 20 using image-guided surgery. In this procedure, a tool 22 is inserted via an incision in the patient's back in order to perform a surgical intervention. Alternatively, the system 10 and the techniques described herein may be used, mutatis mutandis, in other surgical procedures.

Methods for optical depth mapping can generate a three-dimensional (3D) profile of the surface of a scene by processing optical radiation reflected from the scene. In the context of the present description and in the claims, the terms depth map, 3D profile, and 3D image are used interchangeably to refer to an electronic image in which the pixels contain values of depth or distance from a reference point, instead of or in addition to values of optical intensity.

In some embodiments, depth mapping systems can use structured light techniques in which a known pattern of illumination is projected onto the scene. Depth can be calculated based on the deformation of the pattern in an image of the scene. In some embodiments, depth mapping systems use stereoscopic techniques, in which the parallax shift between two images captured at different locations is used to measure depth. In some embodiments, depth mapping systems can sense the times of flight of photons to and from points in the scene in order to measure the depth coordinates. In some embodiments, depth mapping systems control illumination and/or focus and can use various sorts of image processing techniques.

In the embodiment illustrated in FIG. 1, a user of the system 10, such as a healthcare professional 26 (for example, a surgeon performing the procedure), wears the head-mounted unit 28. In some embodiments, the head-mounted unit 28 includes one or more see-through displays 30, for example as described in the above-mentioned U.S. Pat. No. 9,928,629 or in the other patents and applications cited above.

In some embodiments, the one or more see-through displays 30 include an optical combiner. In some embodiments, the optical combiner is controlled by one or more processors 32. In some embodiments, the one or more processors 32 is disposed in a central processing system 50. In some embodiments, the one or more processors 32 is disposed in the head-mounted unit 28. In some embodiments, the one or more processors 32 are disposed in both the central processing system 50 and the head-mounted unit 28 and can share processing tasks and/or allocate processing tasks between the one or more processors 32.

In some embodiments, the one or more see-through displays 30 display an augmented reality image to the healthcare professional 26. In some embodiments, the augmented reality image viewable through the one or more see-through displays 30 is a combination of objects visible in the real world with the computer-generated image. In some embodiments, each of the one or more see-through displays 30 comprises a first portion 33 and a second portion 35. In some embodiments, the one or more see-through displays 30 display the augmented reality image such that the computer-generated image is projected onto the first portion 33 in alignment with the anatomy of the body of the patient 20 that is visible to the healthcare professional 26 through the second portion 35.

In some embodiments, the computer-generated image includes a virtual image of one or more tools 22. In some embodiments, the system 10 combines at least a portion of the virtual image of the one or more tools 22 into the computer-generated image. For example, some or all of the tool 22 may not be visible to the healthcare professional 26 because, for example, a portion of the tool 22 is hidden by the patient's anatomy (e.g., a distal end of the tool 22). In some embodiments, the system 10 can display the virtual image of at least the hidden portion of the tool 22 as part of the computer-generated image displayed in the first portion 33. In this way, the virtual image of the hidden portion of the tool 22 is displayed on the patient's anatomy. In some embodiments, the portion of the tool 22 hidden by the patient's anatomy increase and/or decreases over time or during the procedure. In some embodiments, the system 10 increase and/or decreases the portion of the tool 22 included in the computer-generated image based on the changes in the portion of the tool 22 hidden by the patient's anatomy over time.

Some embodiments of the system 10 comprise an anchoring device (e.g., bone marker 60) for indicating the body of the patient 20. For example, in image-guided surgery and other surgeries that utilize the system 10, the bone marker 60 can be used as a fiducial marker. In some embodiments, the bone marker 60 can be coupled with the fiducial marker. In system 10, for example, the anchoring device is configured as the bone marker 60 (e.g., anchoring device coupled with a marker that is used to register an ROI of the body of the patient 20). In some embodiments, the anchoring device is coupled with a tracking system, with a preoperative or intraoperative CT scan of the ROI. During the procedure, in some embodiments, the tracking system (for example an IR tracking system) tracks the marker mounted on the anchoring device and the tool 22 mounted with a tool marker 40. Following that, the display of the CT image data, including, for example, a model generated based on such data, on the near-eye display may be aligned with the surgeon's actual view of the ROI based on this registration. In addition, a virtual image of the tool 22 may be displayed on the CT model based on the tracking data and the registration. The user may then navigate the tool 22 based on the virtual display of the tool 22 with respect to the patient image data, and optionally, while it is aligned with the user view of the patient or ROI.

According to some aspects, the image presented on the one or more see-through displays 30 is aligned with the body of the patient 20. According to some aspects, misalignment of the image presented on the one or more see-through displays 30 with the body of the patient 20 may be allowed. In some embodiments, the misalignment may be 0-1 mm, 1-2 mm, 2-3 mm, 3-4 mm, 4-5 mm, 5-6 mm, and overlapping ranges therein. According to some aspects, the misalignment may typically not be more than about 5 mm. In order to account for such a limit on the misalignment of the patient's anatomy with the presented images, the position of the patient's body, or a portion thereof, with respect to the head-mounted unit 28 can be tracked. For example, in some embodiments, a patient marker 38 and/or the bone marker 60 attached to an anchoring implement or device such as a clamp 58 or pin, for example, may be used for this purpose, as described further hereinbelow.

When an image of the tool 22 is incorporated into the computer-generated image that is displayed on the head-mounted unit 28 or the HMD unit 70, the position of the tool 22 with respect to the patient's anatomy should be accurately reflected. For this purpose, the position of the tool 22 or a portion thereof, such as the tool marker 40, is tracked by the system 10. In some embodiments, the system 10 determines the location of the tool 22 with respect to the patient's body such that errors in the determined location of the tool 22 with respect to the patient's body are reduced. For example, in certain embodiments, the errors may be 0-1 mm, 1-2 mm, 2-3 mm, 3-4 mm, 4-5 mm, and overlapping ranges therein.

In some embodiments, the head-mounted unit 28 includes a tracking sensor 34 to facilitate determination of the location and orientation of the head-mounted unit 28 with respect to the patient's body and/or with respect to the tool 22. In some embodiments, tracking sensor 34 can also be used in finding the position and orientation of the tool 22 and the clamp 58 with respect to the patient's body. In some embodiments, the tracking sensor 34 comprises an image-capturing device 36, such as a camera, which captures images of the patient marker 38, the bone marker 60, and/or the tool marker 40. For some applications, an inertial-measurement unit 44 is also disposed on the head-mounted unit 28 to sense movement of the user's head.

In some embodiments, the tracking sensor 34 includes a light source 42. In some embodiments, the light source 42 is mounted on the head-mounted unit 28. In some embodiments, the light source 42 irradiates the field of view of the image-capturing device 36 such that light reflects from the patient marker 38, the bone marker 60, and/or the tool marker 40 toward the image-capturing device 36. In some embodiments, the image-capturing device 36 comprises a monochrome camera with a filter that passes only light in the wavelength band of light source 42. For example, the light source 42 may be an infrared light source, and the camera may include a corresponding infrared filter. In some embodiments, the patient marker 38, the bone marker 60, and/or the tool marker 40 comprise patterns that enable a processor to compute their respective positions, i.e., their locations and their angular orientations, based on the appearance of the patterns in images captured by the image-capturing device 36. Suitable designs of these markers and methods for computing their positions and orientations are described in the patents and patent applications incorporated herein and cited above.

In addition to or instead of the tracking sensor 34, the head-mounted unit 28 can include a depth sensor 37. In the embodiment shown in FIG. 2A, the depth sensor 37 comprises a light source 46 and a camera 43. In some embodiments, the light source 46 projects a pattern of structured light onto the region of interest (ROI) that is viewed through the one or more displays 30 by a user, such as professional 26, who is wearing the head-mounted unit 28. The camera 43 can capture an image of the pattern on the ROI and output the resulting depth data to the processor 32 and/or processor 45. The depth data may comprise, for example, either raw image data or disparity values indicating the distortion of the pattern due to the varying depth of the ROI. In some embodiments, the processor 32 computes a depth map of the ROI based on the depth data generated by the camera 43.

In some embodiments, the camera 43 also captures and outputs image data with respect to the markers in system 10, such as patient marker 38, bone marker 60, and/or tool marker 40. In this case, the camera 43 may also serve as a part of tracking sensor 34, and a separate image-capturing device 36 may not be needed. For example, the processor 32 may identify patient marker 38, bone marker 60, and/or tool marker 40 in the images captured by camera 43. The processor 32 may also find the 3D coordinates of the markers in the depth map of the ROI. Based on these 3D coordinates, the processor 32 is able to calculate the relative positions of the markers, for example in finding the position of the tool 22 relative to the body of the patient 20, and can use this information in generating and updating the images presented on head-mounted unit 28.

In some embodiments, the depth sensor 37 may apply other depth mapping technologies in generating the depth data. For example, the light source 46 may output pulsed or time-modulated light, and the camera 43 may be modified or replaced by a time-sensitive detector or detector array to measure the time of flight of the light to and from points in the ROI. As another option, the light source 46 may be replaced by another camera, and the processor 32 may compare the resulting images to those captured by the camera 43 in order to perform stereoscopic depth mapping. These and all other suitable alternative depth mapping technologies are considered to be within the scope of the present disclosure.

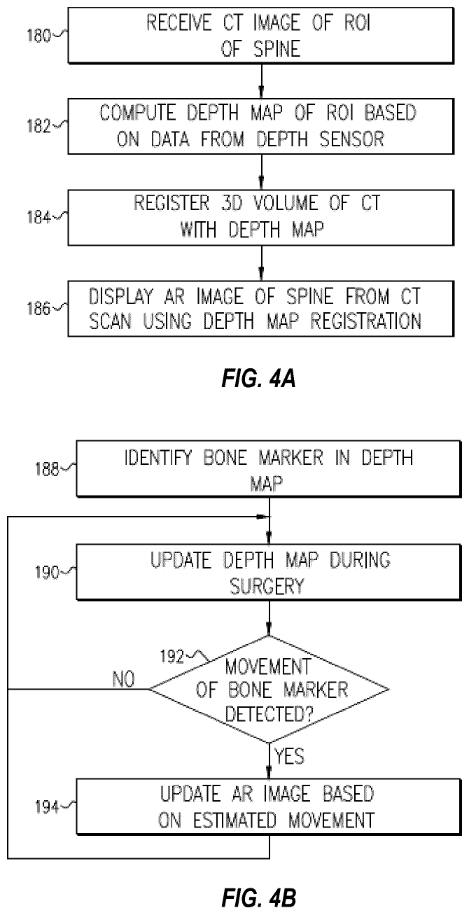

In the pictured embodiment, system 10 also includes a tomographic imaging device, such as an intraoperative computerized tomography (CT) scanner 41. Alternatively or additionally, processing system 50 may access or otherwise receive tomographic data from other sources; and the CT scanner itself is not an essential part of the present system. In some embodiments, regardless of the source of the tomographic data, the processor 32 can computes a transformation over the ROI so as to register the tomographic images with the depth maps that it computes on the basis of the depth data provided by depth sensor 37. The processor 32 can then apply this transformation in presenting a part of the tomographic image on the one or more displays 30 in registration with the ROI viewed through the one or more displays 30. This functionality is described further hereinbelow with reference to FIG. 4A.

In some embodiments, in order to generate and present an augmented reality image on the one or more displays 30, the processor 32 computes the location and orientation of the head-mounted unit 28 with respect to a portion of the body of patient 20, such as the patient's back. In some embodiments, the processor 32 also computes the location and orientation of the tool 22 with respect to the patient's body. In some embodiments, the processor 45, which can be integrated within the head-mounted unit 28, may perform these functions. Alternatively or additionally, the processor 32, which is disposed externally to the head-mounted unit 28 and can be in wireless communication with the head-mounted unit 28, may be used to perform these functions. The processor 32 can be part of the processing system 50, which can include an output device 52, for example a display, such as a monitor, for outputting information to an operator of the system, and/or an input device 54, such as a pointing device, a keyboard, or a mouse, to allow the operator to input data into the system.

Alternatively or additionally, users of the system 10 may input instructions to the processing system 50 using a gesture-based interface. For this purpose, for example, the depth sensor 37 may sense movements of a hand 39 of the healthcare professional 26. Different movements of the professional's hand and fingers may be used to invoke specific functions of the one or more displays 30 and of the system 10.

In general, in the context of the present description, when a computer processor is described as performing certain steps, these steps may be performed by external computer processor 32 and/or computer processor 45 that is integrated within the head-mounted unit. The processor or processors carry out the described functionality under the control of suitable software, which may be downloaded to system 10 in electronic form, for example over a network, and/or stored on tangible, non-transitory computer-readable media, such as electronic, magnetic, or optical memory.

FIG. 2B is a schematic pictorial illustration showing details of a head-mounted AR display (HMD) unit 70, according to another embodiment of the disclosure. HMD unit 70 may be worn by the healthcare professional 26, and may be used in place of the head-mounted unit 28 (FIG. 1). HMD unit 70 comprises an optics housing 74 which incorporates a camera 78, and in the specific embodiment shown, an infrared camera. In some embodiments, the housing 74 comprises an infrared-transparent window 75, and within the housing, i.e., behind the window, are mounted one or more, for example two, infrared projectors 76. One of the infrared projectors and the camera may be used, for example, in implementing a pattern-based depth sensor.

In some embodiments, mounted on housing 74 are a pair of augmented reality displays 72, which allow professional 26 to view entities, such as part or all of patient 20, through the displays, and which are also configured to present to surgeon images or any other information. In some embodiments, the displays 72 present planning and guidance information, as described above.

In some embodiments, the HMD unit 70 includes a processor 84, mounted in a processor housing 86, which operates elements of the HMD unit. In some embodiments, an antenna 88, may be used for communication, for example with processor 32 (FIG. 1).

In some embodiments, a flashlight 82 may be mounted on the front of HMD unit 70. In some embodiments, the flashlight may project visible light onto objects so that professional is able to clearly see the objects through displays 72. In some embodiments, elements of the HMD unit 70 are powered by a battery (not shown in the figure), which supplies power to the elements via a battery cable input 90.

In some embodiments, the HMD unit 70 is held in place on the head of professional 26 by a head strap 80, and the professional may adjust the head strap by an adjustment knob 92.

In some embodiments, the HMD may comprise a visor, which includes an AR display positioned in front of each eye of the professional and controlled by the optical engine to project AR images into the pupil of the eye.

In some embodiments, the HMD may comprise a light source for tracking applications, comprising, for example, a pair of infrared (IR) LED projectors, configured to direct IR beams toward the body of patient 20. In some embodiments, the light source may comprise any other suitable type of one or more light sources, configured to direct any suitable wavelength or band of wavelengths of light. The HMD may also comprise one or more cameras, for example, a red/green/blue (RGB) camera having an IR-pass filter, or a monochrome camera configured to operate in the IR wavelengths. The one or more camera s may be configured to capture images including the markers in system 10 (FIG. 1).

In some embodiments, the HMD may also comprise one or more additional cameras, e.g., a pair of RGB cameras. In some embodiments, each RGB camera may be configured to produce high-resolution RGB (HR RGB) images of the patient's body, which can be presented on the AR displays. Because the RGB cameras are positioned at a known distance from one another, the processor can combine the images to produce a stereoscopic 3D image of the site being operated on.

In some embodiments, the HMD light source 46 (FIG. 2A) may comprises a structured light projector (SLP) which projects a pattern onto an area of the body of patient 20 on which professional 26 is operating. In some embodiments, light source 46 comprises a laser dot pattern projector, which is configured to apply to the area structured light comprising a large number (typically between hundreds and hundreds of thousands) of dots arranged in a suitable pattern. This pattern serves as an artificial texture for identifying positions on large anatomical structures lacking fine details of their own, such as the skin and surfaces of the vertebrae. In some embodiments, one or more cameras 43 capture images of the pattern, and a processor, such as processor 32 (FIG. 1), processes the images in order to produce a depth map of the area. In some embodiments, the depth map is calculated based on the local disparity of the images of the pattern relative to an undistorted reference pattern, together with the known offset between the light source and the camera.

In some embodiments, the projected pattern comprises a pseudorandom pattern of dots. In this case, clusters of dots can be uniquely identified and used for disparity measurements. In the present example, the disparity measurements may be used for calculating depth and for enhancing the precision of the 3D imaging of the area of the patient's body. In some embodiments, the wavelength of the pattern may be in the visible or the infrared range.

In some embodiments, the system 10 (FIG. 1) may comprise a structured light projector (not shown) mounted on a wall or on an arm of the operating room. In such embodiments, a calibration process between the structured light projector and one or more cameras on the head-mounted unit or elsewhere in the operating room may be performed to obtain the 3D map.

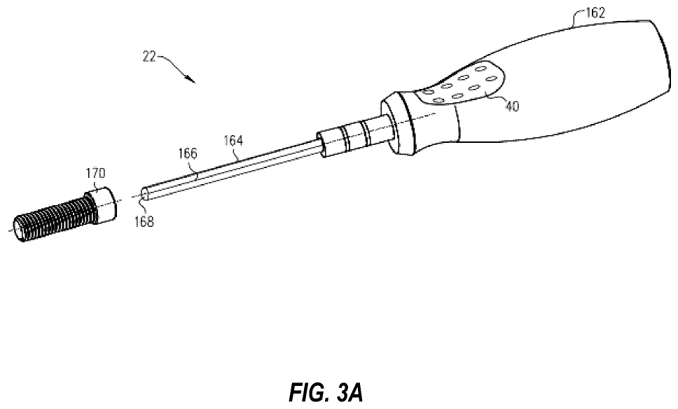

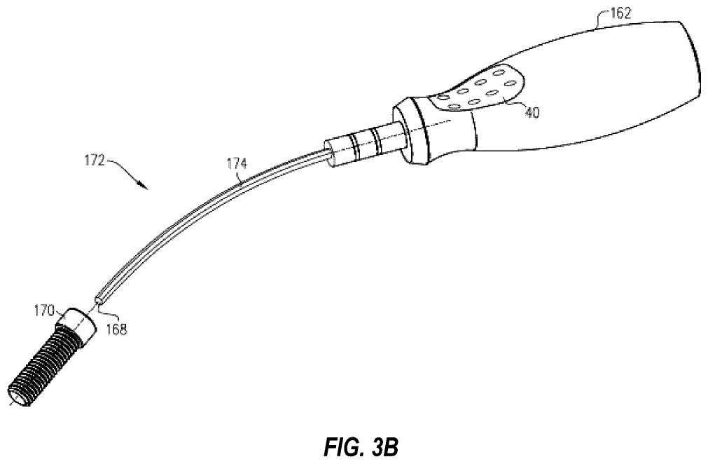

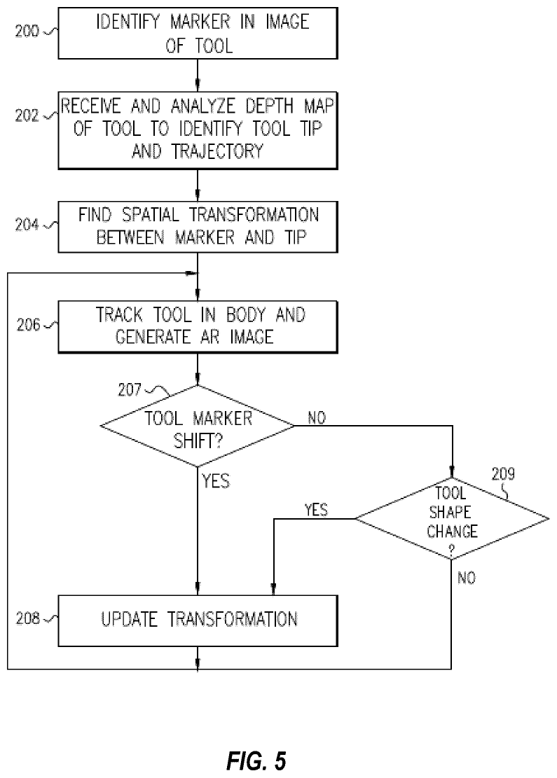

FIG. 3A is a schematic pictorial illustration showing details of the tool 22, in accordance with an embodiment of the disclosure. In some embodiments, the tool 22 comprises a handle 162 and a shaft 164. In some embodiments, the marker 40, containing a predefined pattern, is disposed on the handle 162 in a fixed spatial relation to shaft 164. Alternatively, marker 40 may protrude outward from tool 22 to ensure that it is visible to tracking sensor 34 and/or camera 43 (FIG. 2A). Additionally or alternatively, the depth sensor 37 generates depth data with respect to tool 22. Uses of the depth data in tracking and displaying images of tool 22 are described further hereinbelow with reference to FIG. 5.

In some embodiments, based on the image information provided by tracking sensor 34 and/or camera 43, processor 32 (or processor 45) is able to compute the position (location and orientation) of marker 40 and to track changes in the position of the marker during the surgical procedure. In some embodiments, using the computed position and the known spatial relation of marker 40 to shaft 164, processor 32 is thus able to find the orientation angle of a longitudinal axis 166 of shaft 164 and the location of a distal end 168 of the shaft, even when the shaft is inside the patient's body. On this basis, processor 32 generates an image of tool 22, including shaft 164, on displays 30 in registration with the ROI viewed through the displays.

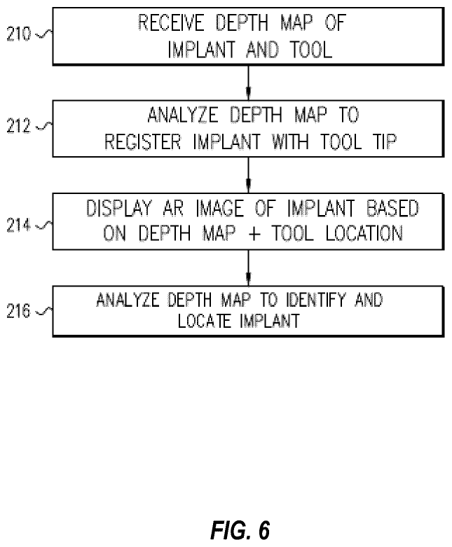

In the example shown in FIG. 3A, tool 22 is used in inserting a surgical implant 170 into the body of patient 20. For the purpose of insertion, implant 170 is mounted on distal end 168 of shaft 164. In one embodiment, the depth sensor 37 captures depth data with respect to both tool 22 and implant 170. In some embodiments, the processor 32 uses the depth data in generating and displaying images of both the tool and the implant, as described further hereinbelow with reference to FIG. 6.

FIG. 3B is a schematic pictorial illustration showing details of tool 172, in accordance with an alternative embodiment of the disclosure. Tool 172 is similar to tool 22 and includes similar components, which are labeled with the same indicator numbers. In contrast to tool 22, however, tool 172 has a curved shaft 174. The curvature of shaft 174 may be fixed, or it may be malleable or otherwise bendable, so that the shape of the shaft may vary from one operation to another or even during a single operation.

To identify the actual shape of shaft 174, processor 32 receives and analyzes a depth map that includes tool 172. The depth map may be captured using any suitable mapping and/or imaging technique, such as the various techniques described above. In some embodiments, the processor 32 segments the depth map to identify the 3D shape of shaft 174 and thus reconstructs a 3D model of tool 172. Based on this 3D model, together with the location of tool marker 40, in some embodiments, the processor 32 is able to compute the location of distal end 168 of shaft 174 even when the distal end is hidden from sight inside the patient's body. In some embodiments, the processor 32 can use the depth data in generating and displaying images of both tool 172 and implant 170, which professional 26 inserts using tool 172.

### Methods for Image-Guided Surgery Using Depth Sensing

FIG. 4A is a flow chart that schematically illustrates a method for image-guided surgery, in accordance with an embodiment of the disclosure. This method, as well as the methods presented in the figures that follow, is described here with reference to the elements of system 10 (as shown in FIGS. 1, 2A and 3A) for the sake of convenience and clarity. Alternatively, the principles of these methods may be applied, mutatis mutandis, in other sorts of augmented reality systems with suitable head-mounted units and depth sensing capabilities, including specifically the head-mounted units shown in FIG. 2B, as well as in the system shown in FIG. 8.

In some embodiments, the processing system 50 receives a 3D tomographic image, such as a CT image, of at least an ROI within the body, at an image input step 180. This image may be based on a scan or scans performed before and/or in the course of the surgical procedure. In some embodiments during the procedure, depth sensor 37 generates depth data with respect to the ROI, and processor 32 applies the depth data in computing a depth map of the ROI, at a depth mapping step 182. For example, the depth map may include the spine of patient 20, which may be exposed in a surgical procedure.

In some embodiments, the processor 32 registers the 3D volume of the tomographic image from the CT scan with the depth map, at a registration step 184. For this purpose, for example, processor 32 extracts 3D point clouds from both the tomographic image and the depth map and then computes a transformation to register one with the other. For instance, during surgery on the spine, the processor may compute the transformation by registering the spine in the depth map with the spine appearing in the tomographic image. Various methods for volume analysis may be used to perform the registration. In one embodiment, processor 32 computes triangular meshes based on the tomographic and depth data and then matches the features of the two meshes.

In some embodiments, the processor 32 applies the transformation computed at step 184 in presenting a part of the tomographic image on displays 30 of head-mounted unit 28 in registration with the ROI viewed through the displays, at a display step 186. In accordance with several embodiments, to display a tracked tool together with the display of the ROI in step 186, the depth sensor 37 (e.g., using cameras of the depth sensor 37) must be calibrated with the tracking system (e.g., tracking sensor 34). In some embodiments, the processor 32 may use the position of patient marker 38, as detected by camera 43 and/or tracking sensor 34, in improving the registration. For example, by identifying the position of patient marker 38 in the depth map, processor 32 may recognize where professional 26 is standing relative to the body of patient 20. In some embodiments, the processor will then rotate the AR image that is presented on head-mounted unit 28 automatically according to the position of the head-mounted unit relative to the patient marker.

In some embodiments, the processor 32 continues to receive and process depth data output by depth sensor 37 during the surgical procedure. In some embodiments, the processor identifies changes in anatomical structures in the body of patient 20 during the procedure, based on the corresponding changes in the depth map. For example, processor 32 can detect that a part of a bone has been cut or that an implant has been inserted or removed. On this basis, processor 32 can modify the AR image presented on the display of head-mounted unit 28 to reflect the identified change.

FIG. 4B is a flow chart that schematically illustrates a method for image-guided surgery, in accordance with another embodiment of the disclosure. This embodiment builds on the method of FIG. 4A and applies the method in detecting movement of the bone marker 60, and/or movement of a bone anchoring implement or device such as the bone clamp 58 relative to the bone to which it is attached, such as the patient's spine. Identification and measurement of such movement is important since it can affect the overall accuracy of system 10.

In some embodiments, the processor 32 identifies the location of bone marker 60 and/or the bone anchoring device in an initial depth map that is generated using depth sensor 37, at a bone marker and/or bone anchoring device identification step 188. In some embodiments, the depth map may be registered with the tomographic image of the patient's spine using the procedure of step 184 of FIG. 4A, as described above. In some embodiments, the processor 32 can thus use the depth data in accurately calculating the position of bone marker 60 and/or bone clamp 58 relative the patient's spine.

Subsequently, in the course of the surgery in some embodiments, the depth sensor 37 continues to capture depth data, and processor periodically updates the depth map, at a depth update step 190. In one embodiment, processor identifies bone marker 60 and/or bone clamp 58 in the updated depth map and checks whether the location of the clamp has shifted relative to the location of spine, as derived from the tomographic image, at a movement detection step 192. When the location of the clamp in the updated depth map has changed, processor 32 takes a corrective action. For example, the processor 32 may modify the image presented on display 30 to reflect this change, at an image update step 194. Additionally or alternatively, the processor 32 may issue a warning to the healthcare professional 26 that one of the markers has moved, such as the bone marker 60 or the patient marker 38, so that the professional can take corrective action if needed.

FIG. 5 is a flow chart that schematically illustrates a method for image-guided surgery, in accordance with yet another embodiment of the disclosure. In some embodiments, the method uses both the 3D shape of the tool 22, which processor 32 derives from the depth data provided by depth sensor 37, and the position of the tool marker 40. The position of the tool marker 40 may be measured by tracking sensor 34, or it may alternatively be extracted from images captured by camera 43 of the depth sensor 37. In either case, in some embodiments, with all the elements used in finding the position of marker 40 and generating depth data with respect to tool 22 being fixed to the head-mounted unit 28 in a known spatial relation, the position of the marker will be consistently registered with the depth map.

In some embodiments, the method of FIG. 5 includes two calibration phases: The first phase includes performing initial calibration prior to the procedure. The second phase provides continuous calibration to update the initial calibration during the procedure. There may be two manners in which the initial calibration may be performed and then updated. In both manners, tool 22 and marker 40 (while the marker is mounted on the tool) are modeled by using depth sensing, for example to identify the trajectory and tip of the tool and the position of the marker with respect to the tool. In some embodiments, the processing system 50 then identifies the tool and marker and determines their relative locations. This function can be carried out using computer vision, for example using function of detection and segmentation, and optionally by applying machine learning techniques, including deep learning. Alternatively or additionally, tracking sensor 34 is used to identify the marker in the 3D model, in which case computer vision may be used only to identify the tool. In the continuous calibration update phase, either or both methods may be used, as well. Although the method shown in FIG. 5 and described below includes both calibration phases, aspects of the method may be applied only in the initial calibration without updating. Furthermore, although some steps in FIG. 5 and in the description below make use of tool marker 40, the method may alternatively be carried out, mutatis mutandis, using only depth sensing without reliance on a tool marker.

In some embodiments, the processor 32 identifies tool marker 40 in an image of tool 22, at a marker identification step 200. As noted earlier, the image may be provided either by tracking sensor 34 or by camera 43. In some embodiments, the processor 32 analyzes the image to derive the position of the tool marker.

In some embodiments, the processor 32 also processes depth data provided by depth sensor 37 in order to generate a depth map of tool 22, possibly along with other elements in the ROI, at a depth mapping step 202. In some embodiments, the processor 32 analyzes the shape of tool 22 in the depth map, for example using techniques of 3D image segmentation and/or machine learning that are known in the art. On this basis, for example, the processor 32 may identify shaft 164 (FIG. 3A) and is thus able to find the orientation of axis 166, corresponding to the “trajectory” of tool 22, and the location of distal end or tip 168. When the shaft of the tool is not straight, for example as in tool 172 (FIG. 3B), processor 32 may use the information provided by the depth map in modeling the tool and finding the location and orientation of the distal tip.

Based on the marker position found at step 200 and the depth map analysis of step 202, in some embodiments, the processor 32 computes a spatial transformation between the position of tool marker 40 and the location and orientation of shaft 164, at a transformation step 204. This transformation can be computed on the basis of the position of the marker and the depth data, without requiring prior knowledge of the shape of tool 22 or of the precise position of marker 40 relative to the other parts of the tool. Alternatively, a priori information regarding the position of the marker on the tool may be used to improve the accuracy of the transformation.

Using the tool marker 40 and/or updated depth data, the processor 32 tracks or may receive tracking information about the position of the tool 22 as professional 26 manipulates the tool with the shaft 164 inserted into the body (and thus not visible to depth sensor 37), at a tracking step 206. Using the tracked position and the spatial transformation computed at step 204, processor 32 generates an image of tool 22 on display 30, in registration with the ROI viewed through the display. The image specifically shows shaft 164, and particularly distal end 168, which when placed inside the patient's body may not be directly visible to professional 26.

As noted earlier, although tool marker 40 is shown in FIG. 3 as a part of handle 162, in other embodiments the tool marker may be fixed externally to the tool. In this case, the tool marker is liable to shift relative to the tool. Processor 32 analyzes the depth data provided by depth sensor 37 in order to calibrate the position of the tool marker relative to the tool. Thus, according to some embodiments, the processor may optionally update this analysis periodically at step 206 during the surgical procedure and checks the position to determine whether the tool marker has shifted relative to the tool, at a shift detection step 207. If so, processor 32 recalibrates the tool position to compensate for the shift, and based on the new calibration, updates the transformation that was computed previously (at step 204), at a transformation update step 208. The image presented on display 30 is modified accordingly.

It may also occur that the shape of tool 22 or tool 172 changes in the course of the surgical procedure. For example, the bending angle of shaft 174 may change, or the shaft may even break. Processor 32 analyzes the depth data provided by depth sensor 37 in order to determine whether the shape of the tool has changed, at a shape change detection step 209. If so, processor 32 recalibrates the tool shape to compensate for the change, and based on the new calibration, updates the transformation that was computed previously (at step 204), at transformation update step 208. The image of the tool on display 30 is updated, as well.

FIG. 6 is a flow chart that schematically illustrates a method for image-guided surgery, in accordance with a further embodiment of the disclosure. This embodiment extends the method of FIG. 5 to deal with the shape and position of implant 170, in addition to tool 22. Although FIG. 3 shows implant 170 as a screw (for example a pedicle screw), the present method is equally applicable to implants of other types, such as stents, cages, and interbody devices. The present method is advantageous in that it enables processor 32 to extract the shapes of implants without requiring prior knowledge of the implant characteristics, and to use the extracted shapes in presenting virtual images of the implants on display 30.

As an initial step, before inserting implant 170 into the body of patient 20, processor 32 generates (or accesses if depth maps were previously generated) depth maps of both implant 170 and tool 22, at a depth mapping step 210. For this purpose, for example, a user of system 10 may be prompted to operate depth sensor 37 to capture depth data with respect to implant 170 by itself and also with respect to tool 22 with implant 170 mounted on distal end 168. Processor 32 analyzes the depth data to identify the shape of implant 170. Based on this shape and on the depth map of implant 170 mounted on tool 22, processor 32 computes a spatial transformation between the position of the tip of tool 22 and the location and orientation of implant 170, at a registration step 212. A spatial transformation between the position of marker 40 and the location and orientation of implant 170 may be computed therefrom. Alternatively or additionally, a transformation between marker 40 and the tip of tool 22 may have been computed at an earlier phase, for example at a calibration phase as described above.

Following these steps, professional 26 uses tool 22 to insert implant 170 into the body of patient 20. Processor 32 tracks the position of marker 40 as professional 26 manipulates shaft 164 of tool 22 within the body. According to some aspects, such tracking information is accessed by processor 32. Using the tracked position, the spatial transformation computed at step 212, and the shape of the implant that was identified at step 210, processor 32 generates a virtual image on display 30 showing the implant within the body and/or its navigation, in registration with the ROI viewed through the display, at an image generation step 214.

After implant 170 has been inserted in place, processor 32 may continuing processing the depth data provided by depth sensor 37 in order to identify and locate the implant, at an implant tracking step 216. For example, processor 32 may identify the head of a screw in the depth map as a cue to the location and orientation of the screw. The display on head-mounted unit 28 can be modified to show the implant in the proper location. The information regarding the location and orientation of the implant can also be useful in planning subsequent stages of the procedure, such as insertion of rods between the screws.

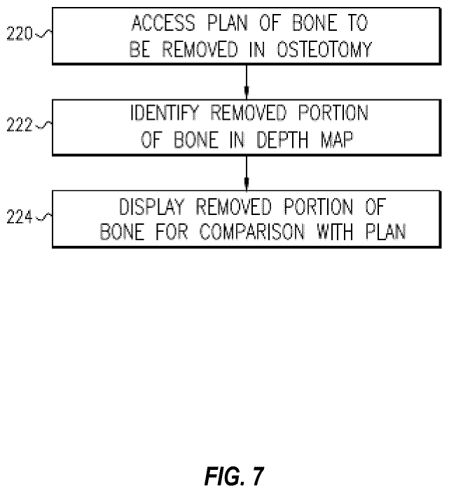

FIG. 7 is a flow chart that schematically illustrates a method for image-guided surgery, in accordance with an additional embodiment of the disclosure. This method is applicable particular in osteotomies, to assist in visualizing the portion of a bone that is removed in the procedure. In such a procedure, processing system 50 accesses a plan made by the surgeon to remove a certain volume of the bone in question, for example a part of one or more vertebrae, at a planning step 220. The present method can assist the surgeon in comparing the volume of the bone that has been cut out to the planned volume.

For this purpose, prior to cutting of the bone, the processor 32 processes depth data generated by depth sensor 37 so as to identify the 3D shape of the bone, for example by generating a point cloud. The 3D cutting plan can be overlaid on the patient's actual anatomy (without displaying patient image data), optionally in a semi- or partially-transparent manner. For example, the top plane of the plan can be overlaid on the patient in alignment with and oriented according to the patient anatomy. The plan may be used as a guide for cutting.

As the surgeon cuts the bone, the outline or indication of a plane of the bone to be removed according to plan changes according to the cutting tool tip location, for example based on depth of penetration. The plan outline or plane can be displayed from a point of view defined by the tool orientation, as determined by tracking the tool tracking. This mode is especially compatible with a “Tip View” mode, in which the patient spine model displayed on the near-eye display, generated based on CT data, changes according to the tool tip location and such that the upper surface of the model is the upper plane defined by the tool orientation and tip location. For example, the model may be cut up to a plane orthogonal to the tool trajectory or longitudinal orientation such that only a portion of the model is displayed.

It is also possible to display or indicate what was done already or the portion of the procedure already performed (e.g., what portion of the bone was already cut and what portion of the bone is left to be cut). The already-cut portion may be indicated on the plan, for example in a different color, or augmented on the image or on the actual patient anatomy or the plan may be updated to show only the remaining bone, and may also show when a portion of the bone was not cut according to plan. Tracking of the cutting may be performed based on tool tip tracking or by depth sensing.

To track cutting using depth sensing, a first depth image of the bone is captured prior to cutting. During the cutting, additional depth images are captured. The capturing may be performed on user request or automatically, continuously or at predefined time interval. Each depth image is compared to the previous one, and processor 32 identifies whether a bone portion was removed or not, i.e., whether cutting was performed. When a difference in the bone volume is identified, the portion of bone that was removed may be indicated on or compared to the plan and displayed to the user. The depth sensor and the tracking system may be calibrated for this purpose. Alternatively, or additionally, the depth maps may be registered with the CT model, for example using feature matching. The calibration and registration process may allow comparison between the different depth maps, the CT model, and the plan.

After the bone has been cut, in some embodiments, the processor 32 accesses and processes new depth data in order to identify the modified 3D shape of the bone. Based on the difference between the 3D shapes, processor 32 identifies the portion of the bone that was removed, at an excision identification step 222. In some embodiments, the processor 32 can then display an image showing the part of the bone that was removed in the surgical procedure, at a display step 224. The surgeon can compare this image to the plan in order to verify that the osteotomy was completed according to plan. In some embodiments, the processor 32 can display both images, i.e., of the removed bone volume and of the planned volume, simultaneously to facilitate comparison between the two. The images may be displayed adjacent to or overlaid on one another.

Alternatively, processor 32 may display to the surgeon only the removed portion of the bone, without comparison to a plan. The processor may thus demonstrate the removed volume and assist in confirming the procedure or in deciding whether a correction or a further operation is required, for example. Additionally or alternatively, in cases in which an implant is to be placed in the body in place of the removed portion of the bone, the surgeon and/or processor 32 may use the model of the removed bone portion to select a suitable implant or to determine whether a particular implant is suitable. On this basis, a suitable implant may be selected from a database, for example. When comparing the removed bone volume to a specific implant, size data may be provided with respect to the implant, or it may be generated using the depth sensing techniques described above.

In some embodiments, the processor 32 generates a model of the removed portion of the bone, which can be used as an input for an implant printing device (such as a 3D printer). The implant may thus be designed based on the model of the removed portion of bone.

In one embodiment, the image and/or model of the ROI that is presented on the AR display, such as the patient spine model, dynamically changes according to the cutting performed and based on the tracking of cutting that is described above. Thus, if a drill is used, for example, then holes may be formed in the model correspondingly, based on tool tracking, for example. The model may be updated in this manner during the procedure and during cutting. At the end of the procedure, the user may be presented with a model showing the entire bone portion removed.

### Alternative Embodiment

FIG. 8 is a schematic pictorial illustration of a system 240 for image-guided surgery, in accordance with an alternative embodiment of the invention. System 240 is similar in design and operation to system 10 (FIG. 1), except that system 240 does not use the sorts of patient marker and clamp marker that were described above, with the accompanying tracking system. Rather, system 240 comprises an array of fiducial markers 242, which are fixed to the back of patient 20, for example using a suitable adhesive. Fiducial markers 242 comprises 3D elements, such as metal beads, which are both radiopaque and visible to cameras, such as camera 43 (FIG. 2A).

Prior to the surgical procedure, markers 242 are fixed to the back of patient 20, and a CT scan of the patient is performed, for example using CT scanner 41 (FIG. 1). Markers 242 will appear in the resulting CT images along with the patient's skeleton. During the surgery, depth sensor 37 on head-mounted unit 28 captures depth maps of the patient's back, including markers 242. Processor 32 analyzes the depth maps to find the 3D coordinates of markers 242. The processor then matches the locations of markers 242 in the CT image data with the 3D coordinates of the markers in the depth map, and thus derives the appropriate transformation to register the CT image data with the actual patient anatomy viewed by professional 26. Based on this registration, processor 32 presents parts of the CT images on displays 30, overlaid on the patient anatomy.

The other features and applications of depth mapping in image-guided surgery that are described above may likewise be applied, mutatis mutandis, in system 240.

Although the drawings and embodiments described above relate specifically to surgery on the spine, the principles of the present disclosure may similarly be applied in other sorts of surgical procedures, such as operations performed on the cranium and various joints, as well as dental surgery. It will thus be appreciated that the embodiments described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

As used herein “generate” or “generating” may include specific algorithms for creating information based on or using other input information. Generating may include retrieving the input information such as from memory or as provided input parameters to the hardware performing the generating. Once obtained, the generating may include combining the input information. The combination may be performed through specific circuitry configured to provide an output indicating the result of the generating. The combination may be dynamically performed such as through dynamic selection of execution paths based on, for example, the input information, device operational characteristics (for example, hardware resources available, power level, power source, memory levels, network connectivity, bandwidth, and the like). Generating may also include storing the generated information in a memory location. The memory location may be identified as part of the request message that initiates the generating. In some implementations, the generating may return location information identifying where the generated information can be accessed. The location information may include a memory location, network locate, file system location, or the like.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general purpose computers. For example, the methods described herein may be performed by the processors 32, 45 described herein and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As it is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated. While the embodiments provide various features, examples, screen displays, user interface features, and analyses, it is recognized that other embodiments may be used.

What is claimed is:

1. A system for image-guided surgery, the system comprising:
a head-mounted unit comprising:
a see-through augmented reality (STAR) display;
a depth sensor configured to generate depth data with respect to a region of interest (ROI) on a body of a patient viewed through the STAR display by a user wearing the head-mounted unit and with respect to a surgical item when the surgical item is placed within a field of view of the depth sensor, wherein the surgical item comprises a first marker containing a predefined pattern disposed on the surgical item; and
a processor configured to:
process the depth data to identify a shape of the surgical item;
compute, responsively to the shape, a first spatial transformation between a position of the first marker and a location and orientation of the surgical item;
track the position of the first marker as the user manipulates the surgical item; and
using the tracked position, the first spatial transformation, and the identified shape, generate an image of the surgical item on the STAR display in registration with the ROI viewed through the STAR display.

2. The system of claim 1, wherein to generate the depth data, the depth sensor is configured to generate depth data with respect to a hand of a user of the head-mounted unit, and wherein the processor is configured to process the depth data to detect a gesture made by the hand and to control a function of the system responsive to a gesture detected by the processor.

3. The system of claim 1, wherein:
the head-mounted unit further comprises a visor,
the STAR display is operably coupled to the visor and positioned in front of each eye of the user, and
an optical engine controls the STAR display to project augmented reality images into a pupil of each eye.

4. The system of claim 1, wherein the surgical item further comprises a tool with a shaft, wherein the first marker protrudes outward from the tool such that it is visible to the depth sensor when a portion of the tool is inserted into the body of the patient.

5. The system of claim 1, wherein the head-mounted unit further comprises an inertial-measurement unit configured to sense movement of a user's head.

6. The system of claim 1, wherein the depth sensor comprises:
a light source configured to output time-modulated light; and
a time-sensitive detector operable to measure a time of the light source to and from points in the ROI.

7. The system of claim 1, wherein the depth sensor comprises a pair of cameras configured for stereoscopic depth mapping.

8. The system of claim 1, wherein the surgical item further comprises a tool with a shaft, wherein the predefined pattern of the first marker is disposed on the tool in a fixed spatial relation to the shaft.

9. The system of claim 8, wherein the shaft has a curved shape, and wherein to process the depth data, the processor is configured to process the depth data to reconstruct a three-dimensional (3D) model of the curved shape and generate the image of the tool based on the 3D model.

10. The system of claim 8, wherein to process the depth data, the processor is configured to process the depth data to detect a change in a shape of the tool and to update the image of the tool on the STAR display responsively to the change in the shape.

11. The system of claim 1, wherein the surgical item further comprises:
a tool with a shaft; and
an implant mounted on the shaft and configured to be inserted into the body of the patient using the tool,
wherein the predefined pattern of the first marker is disposed on the tool in a fixed spatial relation to the shaft.

12. The system of claim 11, wherein the processor is configured to generate a depth map of both the implant and the tool prior to insertion of the implant into the body of the patient.

13. The system of claim 12, wherein the processor is further configured, based on the depth data and an exposed feature of the implant in the body of the patient, to generate an image of the implant on the STAR display in registration with the ROI viewed through the STAR display.

14. The system of claim 1, wherein to generate the depth data, the depth sensor is configured to generate the depth data with respect to a second marker that is attached to the body of the patient, and wherein the processor is configured to apply the depth data in calculating a position of the surgical item relative to the body.

15. The system of claim 14, wherein the processor is further configured to:
receive a three-dimensional (3D) tomographic image of the body of the patient;
compute a depth map of the ROI based on the depth data, the depth map including the ROI on the body of the patient and including an anatomical feature of the patient, the second marker attached to the body of the patient, and the surgical item;
compute a second transformation over the ROI to register the 3D tomographic image with the depth map by registering the anatomical feature in the depth map with the anatomical feature appearing in the 3D tomographic image; and
apply the second transformation in presenting a part of the tomographic image on the STAR display in registration with the ROI viewed through the STAR display.

16. The system of claim 1, further comprising a tracking sensor disposed on the head-mounted unit in a known spatial relation to the depth sensor and configured to detect the position of the first marker.

17. The system of claim 16, wherein the tracking sensor comprises:
a light source mounted on the head-mounted unit; and
an image-capturing device,
wherein the light source is configured to irradiate a field of view such that light reflects from the first marker toward the image-capturing device.

18. The system of claim 17, wherein the image-capturing device comprises a monochrome camera with a filter configured to pass light in a wavelength band of the light source.

19. A computer-implemented method for image-guided surgery, comprising:
using a head-mounted unit that includes a see-through augmented reality (STAR) display and a depth sensor, generating depth data with respect to a region of interest (ROI) on a body of a patient that is viewed through the STAR display by a user wearing the head-mounted unit and with respect to a surgical item when the surgical item is placed within a field of view of the depth sensor, wherein the surgical item comprises a marker containing a predefined pattern disposed on the surgical item;
processing the depth data to identify a shape of the surgical item;
computing, responsively to the shape, a spatial transformation between a position of the marker and a location and orientation of the surgical item;
tracking the position of the marker as the user manipulates the surgical item; and
using the tracked position, the spatial transformation, and the identified shape, generating an image of the surgical item on the STAR display in registration with the ROI viewed through the STAR display.

20. A non-transitory computer readable medium storing instructions for image-guided surgery, which when executed by at least one processor, cause the at least one processor to perform operations comprising:
using a head-mounted unit that includes a see-through augmented reality (STAR) display and a depth sensor, generating depth data with respect to a region of interest (ROI) on a body of a patient that is viewed through the STAR display by a user wearing the head-mounted unit and with respect to a surgical item when the surgical item is placed within a field of view of the depth sensor, wherein the surgical item comprises a marker containing a predefined pattern disposed on the surgical item;
processing the depth data to identify a shape of the surgical item;
computing, responsively to the shape, a spatial transformation between a position of the marker and a location and orientation of the surgical item;
tracking the position of the marker as the user manipulates the surgical item; and
using the tracked position, the spatial transformation, and the identified shape, generating an image of the surgical item on the STAR display in registration with the ROI viewed through the STAR display.
